(19) United States
(12) Patent Application Publication
(10) Pub. No.: US 2025/0000895 A1
(43) Pub. Date: Jan. 2, 2025

(54) POLYMERS OBTAINED BY THE REACTION OF A PHENOLIC COMPOUND AND A CARBONYL COMPOUND FOR USE IN THE TREATMENT OR PREVENTION OF A CONDITION ASSOCIATED WITH ONE OR MORE PROTEASE(S)

(71) Applicants: BASF SE, Ludwigshafen (DE); CHRISTIAN-ALBRECHTS-UNIVERSITÄT ZU KIEL, Kiel (DE)

(72) Inventors: Günter Scherr, Ludwigshafen (DE); Ulrich Mrowietz, Kiel (DE)

(21) Appl. No.: 18/696,397

(22) PCT Filed: Sep. 29, 2022
(86) PCT No.: PCT/EP2022/077140

§ 371 Date: Mar. 28, 2024

(30) Foreign Application Priority Data
Sep. 30, 2021 (WO) PCT/EP2021/077013

(51) Int. Cl. A61K 31/795; A01N 47/34; A01P 1/00; A61K 45/06; A61P 31/14; A61P 31/18; A61P 31/22; A61P 35/00; A61P 37/00

(52) U.S. Cl. CPC A61K 31/795; A01N 47/34; A01P 1/00; A61K 45/06; A61P 31/14; A61P 31/18; A61P 31/22; A61P 35/00; A61P 37/00

(57) ABSTRACT

The present invention relates to a condensation product, obtained or obtainable by reaction of phenol, formaldehyde, sulfuric acid and urea, for use in a method for the treatment or prevention of a condition associated with one or more protease(s) in a subject, a pharmaceutical composition comprising such a condensation product and for such a use, a pharmaceutical set comprising such a composition and for such a use, such pharmaceutical composition and such a pharmaceutical set and the non-therapeutic use of such a condensation product, such a pharmaceutical composition or such a pharmaceutical set as a disinfectant.

The present invention relates to a condensation product for use in a method for the treatment or prevention of a condition associated with one or more protease(s) in a subject, a pharmaceutical composition comprising such a condensation product and for such a use, a pharmaceutical set comprising such a composition and for such a use, such pharmaceutical composition and such a pharmaceutical set and the non-therapeutic use of such a condensation product, such a pharmaceutical composition or such a pharmaceutical set as a disinfectant.

Proteases are enzymes that catalyse the breakdown of proteins into smaller peptides or single amino acids, i.e. proteolysis, by cleaving peptide bonds. Proteases are ubiquitously expressed in all tissues and cells of all organisms (including viruses) and are required for a variety of biological processes. Thus, a large amount of different proteases exists.

A classification of the numerous proteases can be drawn by distinguishing the different proteases by their active centre, i.e. specific amino acids, which are of particular importance for the functionality of a protease. Thus, proteases can be classified as serine proteases, cysteine proteases, threonine proteases, aspartic proteases, asparagine proteases, glutamic proteases, metalloproteases (using a metal in the active centre) and other proteases, which cannot be categorized into the aforementioned groups.

By cleaving the peptide bonds, proteases may either abolish a protein's function or digest it to its amino acids or alternatively activate a function of a protein, e.g. as a signal in a signalling pathway. Consequently, proteases are involved in a variety of biological processes.

However, in addition to their required function and due to their involvement in many biological processes, proteases may also cause severe and life-threatening conditions if they are present in amounts higher or lower than the required amount or if their activity is different from the required activity.

This principle may also be considered in case an agent is required, which has an activity against a certain organism for e.g. treating or preventing an infection with a pathogen.

Furthermore, this principle may also be considered in case a subject shows an undesired reaction due to the activity of a protease.

In this regard, infections with a pathogen may be e.g. viral infections, bacterial infections, fungal infections or parasitoses. Since viruses, bacterial, fungi and parasites also make use of proteases, targeting the protease activity in these pathogens may be a possible treatment or prevention of an infection with a virus, bacterium, fungus or parasite.

On the other side, an undesired reaction due to the activity of a protease may be e.g. an allergy, wherein the allergen is a protease, an inflammatory condition, wherein the underlying protease may promote inflammation, an intoxication, wherein a protease is applied to an organism and promotes or influences a biological process in the organism, or a tumor disease, wherein the protease may e.g. promote angiogenesis, which is required for growth of a tumor at a certain size, or degrade the basal membrane and thus facilitate metastatic spread of a tumor.

It has been shown for example by Lin et al., “The Allergen Der p3 from house Dust Mite Stimulates Store-Operated Ca²⁺ Channels and Mast Cell Migration through PAR4 Receptors”, Molecular Cell, 70, 228-241 (2018) that the Der p proteins, which have protease activity, are present in fractions of Dermatophagoides pteronyssinus extract. Der proteins p3, p6 and p9 are serine proteases, Der protein p1 is a cysteine protease. Precisely, it is shown that the serine protease Der p3 is one of the key proteins responsible of mite allergens, e.g. house dust mite allergens, and that the catalytic activity of Der p3 is required for promoting an allergic reaction. Thus, inhibition of Der p3 catalytic activity may be the target for preventing or treating mite allergies such as house dust mite allergy in a subject.

Furthermore, it was shown that the serine protease Proteinase 3 is involved in the pathogenesis of vasculitis, particularly ANCA-associated vasculitis such as GPA (Granulomatosis with polyangiitis, previously known as Wegener's granulomatosis). Particularly in GPA, it is presumed that the anti-neutrophil cytoplasmic antibodies (ANCAs) are responsible for the inflammation in GPA, wherein the ANCAs react with the serine protease Proteinase 3, which is expressed in neutrophils. Thus, inhibiting the binding of ANCAs to Proteinase 3, e.g. by achieving a conformational change of Proteinase 3 can provide a successful prevention or treatment of e.g. GPA.

With regard to intoxications, it is known that snake venoms comprise proteases in large extent. Precisely, enzymes make up to 80 to 90% of viperid and 25 to 70% of elapid venoms. Such snake venoms comprises proteases, especially serine proteases, which cleave e.g. fibrinogen or thrombin and thus influence blood coagulation or which degrade cells, or tissues and thus cause severe damages to an organism. Thus, inhibiting the activity of such proteases can provide an important prevention or treatment of snake venom intoxication in a subject.

Likewise, it was shown that hymenoptera venoms, such as venoms from honey bee or wasps, also comprise proteases, particularly serine proteases, which may either cause intoxications or allergic reactions against these serine proteases. Thus, inhibiting the activity of these serine proteases can provide an important prevention or treatment of hymenoptera venom intoxication in a subject or an allergic reaction against the serine proteases in a subject.

With regard to tumor diseases, it is known that proteases are involved in e.g. the process of angiogenesis and metastasis. When a tumor has grown to a size of approximately 1 to 2 mm³, the tumor requires a capillary supply, promoting access to sufficient oxygen and nutrients. Thus, the formation of new vessels is required, i.e. angiogenesis needs to be induced for the tumor to continue growing. In contrast, if angiogenesis is prevented, the tumor enters a dormant condition (Tumor Dormancy) and does not continue to grow. It is known that the serine protease thrombin is involved in angiogenesis.

Furthermore, it is known that for being a malign tumor, a tumor needs to pass the basal membrane, allowing the tumor to spread, i.e. to metastasize. For passing the basal membrane, tumors typically degrade the basal membrane, which comprises proteins and proteoglycans. Tumors secreting matrix metalloproteases, serine proteases, cysteine proteases, aspartate proteases or threonine proteases are able to degrade the basal membrane, which facilitates or, respectively, allows tumor metastasis. Particularly, Rakashanda et al., “Role of proteases in cancer: A review”, Biotechnol. Mol. Bio. Rev. (2012), 74, 90-101 describes a role of matrix metalloproteases, serine proteases, cysteine proteases, aspartate proteases or threonine proteases in various cancer types. Furthermore, Quiao et al., “Testes-specific protease 50 as an independent risk factor for poor prognosis in patients with non-small cell lung cancer”, Oncology Letters (2018), 15, 8796-8804 describes that TSP50 is produced by tumor cells. Moreover, Rath et al., “Expression of Proteolytic Enzymes by Small Cell Lung Cancer Circulating Tumor Cell Lines”, Cancers (2019), 11, 114 describes that ADAMs 8, 9, S1, and S13; cathepsins A, B, C, D, E, L, S, V, and X/Z/P; MMPs 1, 2, 3, 7, 8, 9, 10, 12, 13; kallikreins 5, 6, 7, 10, 11, 13; neprilysin/CD10, presenilin-1, PC-9, proteinase 3, and uPA are expressed in the tested tumor cell lines.

Thus, inhibiting the activity of such proteases, preferably metalloproteases (preferably matrix metalloproteases), serine proteases, cysteine proteases, aspartate proteases or threonine proteases, or specific proteases or groups of proteases as described herein may provide an important prevention or treatment of a tumor disease.

It was thus an object of the present invention to provide possibilities for interfering with such proteases, preferably with one or more protease(s) selected from the group consisting of metalloproteases (preferably matrix metalloproteases), serine proteases, cysteine proteases, aspartate proteases or threonine proteases and combinations thereof, preferably consisting of metalloproteases, serine proteases and combinations thereof, for providing a prevention and/or treatment of a condition associated with one or more protease(s) in a subject.

Surprisingly it was found that the primary object of the present invention is solved by a condensation product or salt thereof or pharmaceutical composition comprising the condensation product or one or more salt(s) thereof or the condensation product and one or more salt(s) thereof,
wherein the condensation product or salt thereof is obtained or obtainable by reaction of
a1) at least one phenolic compound, and
a2) at least one carbonyl compound,
a3) and optionally: at least one sulfonating agent,
a4) and optionally: urea and/or one or more urea derivative,
wherein the condensation product or salt thereof has a molecular weight Mw in the range of from 500 g/mol to 50,000 g/mol, preferably in the range of from 500 g/mol to 5,000 g/mol,
preferably wherein the pharmaceutical composition further comprises one or more pharmaceutically acceptable carrier(s),
for use in a method for the treatment or prevention of a condition associated with one or more protease(s) in a subject,
wherein the condition is an infection with a pathogen expressing the one or more protease(s) or a reaction of the subject's organism to the one or more protease(s),
wherein the condition is selected from the group consisting of viral infections, fungal infections, bacterial infections, parasitoses, allergies, inflammatory conditions, intoxications and tumor diseases,
wherein the viral infections are selected from the group consisting of Cytomegalovirus (CMV) infections, Human immunodeficiency virus (HIV) infections, Dengue virus infections, Flavivirus infections, West-Nile virus (WNV) infections, Chikungunya virus (CHIKV) infections, Measles morbillivirus (MeV) infections, Respiratory syncital virus (RSV) infections and Foot and Mouth disease virus infections,
wherein the allergies are allergies directed against allergens selected from the group consisting of proteases,
wherein the intoxications are selected from the group consisting of animal-caused intoxications and plant-caused intoxications, preferably animal-caused intoxications selected from snake-caused intoxications, hymenoptera-caused intoxications, jelly fish-caused intoxications and arachnida-caused intoxications.

Preferably, the tumor underlying the tumor disease is selected from tumors secreting one or more proteases selected from the group consisting of metalloproteases (preferably matrix metalloproteases), serine proteases, cysteine proteases, aspartate proteases or threonine proteases, preferably consisting of matrix metalloproteases and serine proteases.

It was surprisingly found that the condensation product to be used according to the invention can inhibit the activity of proteases of pathogens, which are responsible or critically involved in the pathogen's ability to enter and/or reproduce in the subject's organism. Thus, the condensation product to be used according to the invention can be used for preventing an infection with such a pathogen.

It was further surprisingly found that the condensation product to be used according to the invention can inhibit the activity of important proteases in pathogens, which have infected an organism and which express the protease(s), and can thus interfere in required biological pathways of the pathogen. Thus, the condensation product to be used according to the invention can be used for treating an infection with such a pathogen.

Additionally, it was surprisingly found that the condensation product to be used according to the invention can inhibit the activity of proteases, which otherwise provide a reaction of the subject's organism, when exposed to the subject's organism. Thus, the condensation product can be used to prevent such a reaction.

It was further surprisingly found that the condensation product to be used according to the invention can inhibit the activity of proteases, which are exposed to a subject's organism and cause a reaction of the subject's organism. Thus, the condensation product can be used to treat such a reaction.

Surprisingly, it was found that the condensation product according to the present invention may, e.g. by binding to the respective protease, induce a conformational change of the protease, due to which the enzymatic activity of the protease is reduced or blocked. Furthermore, this conformational change can also lead to the situation that the protease is no longer recognised as target of e.g. antibodies or immune cells.

Preferably, the condition is an infection with a pathogen expressing the one or more protease(s).

Preferably, the condition is a reaction of the subject's organism to the one or more protease(s).

It is preferred, that the condition is a viral infection as described herein. Particularly preferably, the protease, with which the condition is associated, is selected from the group consisting of serine proteases, cysteine proteases, threonine proteases, aspartic proteases, glutamic proteases, metalloproteases and asparagine peptide lyases, preferably cysteine proteases, serine proteases and metalloproteases, particularly preferably serine proteases and metalloproteases.

Preferably, the term “Cytomegalovirus” as used herein describes human Cytomegalovirus (hCMV).

Preferably, the term “Respiratory syncital virus” as used herein describes human Respiratory syncital virus (hRSV).

It is preferred, that the condition is a fungal infection as described herein. Particularly preferably, the protease, with which the condition is associated, is selected from the group consisting of serine proteases, cysteine proteases, threonine proteases, aspartic proteases, glutamic proteases, metalloproteases and asparagine peptide lyases, preferably cysteine proteases, serine proteases and metalloproteases, particularly preferably serine proteases and metalloproteases.

It is preferred, that the condition is a bacterial infection as described herein. Particularly preferably, the protease, with which the condition is associated, is selected from the group consisting of serine proteases, cysteine proteases, threonine proteases, aspartic proteases, glutamic proteases, metalloproteases and asparagine peptide lyases, preferably cysteine proteases, serine proteases and metalloproteases, particularly preferably serine proteases and metalloproteases.

It is preferred, that the condition is a parasitosis as described herein. Particularly preferably, the protease, with which the condition is associated, is selected from the group consisting of serine proteases, cysteine proteases, threonine proteases, aspartic proteases, glutamic proteases, metalloproteases and asparagine peptide lyases, preferably cysteine proteases, serine proteases and metalloproteases, particularly preferably serine proteases and metalloproteases.

It is preferred, that the condition is an allergy as described herein. Particularly preferably, the protease, with which the condition is associated, is selected from the group consisting of serine proteases, cysteine proteases, threonine proteases, aspartic proteases, glutamic proteases, metalloproteases and asparagine peptide lyases, preferably cysteine proteases, serine proteases and metalloproteases, particularly preferably serine proteases and metalloproteases.

It is preferred, that the condition is an inflammatory condition as described herein. Particularly preferably, the protease, with which the condition is associated, is selected from the group consisting of serine proteases, cysteine proteases, threonine proteases, aspartic proteases, glutamic proteases, metalloproteases and asparagine peptide lyases, preferably cysteine proteases, serine proteases and metalloproteases, particularly preferably serine proteases and metalloproteases.

It is preferred, that the condition is an intoxication as described herein. Particularly preferably, the protease, with which the condition is associated, is selected from the group consisting of serine proteases, cysteine proteases, threonine proteases, aspartic proteases, glutamic proteases, metalloproteases and asparagine peptide lyases, preferably cysteine proteases, serine proteases and metalloproteases, particularly preferably serine proteases and metalloproteases.

It is preferred, that the condition is a tumor disease as described herein. Particularly preferably, the protease, with which the condition is associated, is selected from the group consisting of serine proteases, cysteine proteases, threonine proteases, aspartic proteases, glutamic proteases, metalloproteases and asparagine peptide lyases, preferably cysteine proteases, serine proteases and metalloproteases, particularly preferably serine proteases and metalloproteases, especially preferably thrombin and matrix metalloproteases.

The term “protease” as used in the context of the present invention refers to enzymes that can cleave covalent chemical bonds within or between proteins and/or peptides. Preferably, the term “protease” as used in the context of the present invention refers to a protease selected from the group consisting of serine proteases, cysteine proteases, threonine proteases, aspartic proteases, glutamic proteases, metalloproteases and asparagine peptide lyases, particularly preferably selected from group consisting of metalloproteases, cysteine proteases and serine proteases, preferably consisting of metalloproteases and serine proteases.

The term “infection” as used herein refers to the condition immediately after one or more pathogens have invaded an organism. Preferably, the term “infection” as used herein refers to the condition that one or more pathogens, preferably one or more viruses and/or one or more bacteria and/or one or more fungi and/or one or more parasites, has/have replicated within the organism. Preferably, the term “infection” refers to a detectable infection, i.e. the condition, in which one or more pathogens, preferably one or more viruses and/or one or more bacteria and/or one or more fungi and/or one or more parasites, has/have already replicated within the organism and have reached a number which can be experimentally verified or detected, as described herein.

The term “pathogen” as used herein refers to any organism or agent that can cause an undesired condition in a subject, such as a disease, a malfunction of the subject's organism, pain or combinations thereof. Preferably, the term “pathogen” refers to an organism selected from the group consisting of viruses, bacteria, parasites, and fungi.

The term “parasite” as used herein refers to an organism, which lives on or inside another organism (host). Preferably, the term “parasite” as used herein refers to ectoparasites and endoparasites. Particularly preferably, the term “parasite” as used herein refers to an organism selected from the group consisting of protozoans, animals (such as worms, lice and mosquitoes) and plants (such as mistletoe, Cuscuta or Orobanchaceae).

Consequently, the term “parasitosis” as used herein describes an infection caused or transmitted by one or more parasite(s). Preferably, the term “parasitosis” as used herein refers to an infectious disease caused by one or more parasite(s), wherein the parasite(s) can cause the disease directly or by toxins, which are produced by the parasite(s).

The term “reaction of the subject's organism” as used herein refers to active reactions, in which the subject's organism is triggered by the one or more protease(s), such as allergies or inflammatory conditions. Furthermore, the term “reaction of the subject's organism” as used herein also refers to passive reactions, in which the protease itself provides a particular effect on the subject's organism, such as intoxications (e.g. in which a protease cleaves a protein in or on the subject's organism and thus causes an undesired condition) or tumor diseases.

Preferably, the bacterial infections as described in the context of the present invention are caused by one or more bacteria selected from the group consisting of Firmicutes, Actinobacteria and Proteobacteria,
wherein the Firmicutes are preferably selected from the group consisting of the genera Staphylococcus, Clostridium, and Enterococcus, and
wherein the Actinobacteria are preferably selected from the group consisting of the family Propionibacteriaceae, particularly preferably selected from the group consisting of the genera Pripionibacterium and Mycobacterium, and
wherein the Proteobacteria are preferably selected from the group consisting of the genera Klebsiella, Morganella, Pseudomona and Stenotrophomona.

Preferably, the fungal infections as described in the context of the present invention are caused by one or more fungi selected from the group consisting of pathogenic yeasts, dermatophytes, and molds,
wherein the pathogenic yeasts are preferably selected from the group consisting of Cryptococcus and Candida, and
wherein the dermatophytes are preferably selected from the group consisting of Ctenomyces, Epidermophyton, Microspirum, Nannizzia, Trichophyton, Paraphyton, Lophophyton, and Arthroderma, and
wherein the molds are preferably selected from the group consisting of Aspergillus, Cryptococcus, Trichophyton, Stachobotrys, and Histoplasma.

Preferably, the term “pathogenic yeast” refers to those yeasts, which are opportunistic pathogens, i.e. these yeasts cause undesired conditions in subjects with a compromised immune system. Examples of such yeasts are yeasts of the genus Cryptococcus or Candida.

Preferably, the parasitoses as described in the context of the present invention are caused by one or more parasites selected from the group consisting of multicellular parasites and unicellular parasites,
wherein multicellular parasites are preferably selected from the group consisting of animals (such as worms, lice and mosquitoes) and plants (such as mistletoe, Cuscuta or Orobanchaceae), and
wherein unicellular parasites are preferably selected from the group consisting of parasitic eukaryotes, preferably protozoa, and parasitic prokaryotes.

The term “unicellular parasite” as used in the context of the present invention refers to single cells as well as to colonies formed by such single cells.

Especially preferably, the parasitoses as described in the context of the present invention are caused by parasites selected from the group consisting of Shistosoma, Plasmodium, Trichinella, Toxoplasma, Trypanosoma, Leishmania, Trichomonas, Entamoeba and Giardia.

The term “infection caused by a bacterium”, can be used interchangeably with the term “bacterial infection”. The same applies accordingly to corresponding terms such as the terms “infection caused by a fungus” and “fungal infection”. Furthermore, the term “infection caused by one or more bacteria” does not refer to one or more single bacterial cells. Instead, the term refers to one or more different types (i.e. phyla, genera, families or the like) of bacteria. The same applies accordingly to corresponding terms such as the terms “infection caused by one or more fungi”.

Typically, methods for detecting a pathogen, which has infected a subject's organism in as described the context of the present invention, are well known to a skilled person. For example, for detecting the pathogen, a sample (such as a blood, tissue, stool, urine) is taken from the subject and is analysed for the presence of the pathogen, e.g. by methods including sequencing, antibody-binding assays, genotyping (involving PCR), detecting the presence (or level) of antibodies, culturing, and combinations thereof.

Preferably, the allergies as described in the context of the present invention are directed to allergens selected from the group consisting of proteases, wherein the allergies are selected from pollen allergies, preferably birch-pollen allergy, and mite allergies, preferably Dermatophagoides allergy.

Additionally or alternatively, it is preferred that the allergens are selected from the group consisting of metalloproteases (preferably matrix metalloproteases), serine proteases, cysteine proteases, aspartate proteases, threonine proteases and combinations thereof, preferably consisting of metalloproteases, cysteine proteases, serine proteases and combinations thereof, preferably consisting of metalloproteases, serine proteases and combinations thereof, preferably selected from the group consisting of pollen proteases and Der proteins, preferably Der p1, Der p2, Der p3, Der p6, Der p7, Der p8, Der p9, Der p10, and combinations thereof. Preferably, the Der protein is Der p3. Preferably, the allergen is Der p3.

Preferably, inflammatory conditions are selected from the group consisting of appendicitis, bursitis, colitis, cystitis, dermatitis, epididymitis, encephalitis, gingivitis, meningitis, myelitis, nephritis, neuritis, pancreatitis, periodontitis, pharyngitis, phlebitis, prostatitis, RSD/CRPS, rhinitis, sinusitis, tendonitis, tonsillitis, urethritis, vasculitis, and vaginitis.

Especially preferably, the inflammatory condition is a vasculitis, particularly preferably ANCA-associated vasculitis.

The term “intoxication” as used in the context of the present invention refers to a subject's exposition to one or more external substance(s), excluding organisms, at an amount that causes negative effects to the subject, such as illness, disease, malfunction of the subject's organism, pain, inflammation, and combinations thereof. Preferably, the or one or more or all external substance(s) is/are or comprises a protease, preferably selected from the group consisting of metalloproteases, cysteine proteases and serine proteases, preferably consisting of metalloproteases and serine proteases.

The term “animal-caused intoxication” as used herein refers to an intoxication, wherein the one or more external substance(s) exposed to the subject were produced by an animal and transferred from the animal to the subject, e.g. by application during a bite or sting of said animal.

Preferably, the animal is a snake, a honey bee, a wasp, a jelly fish, a scorpion or a spider. Consequently, it is preferred that the animal-caused intoxications are selected from snake-caused intoxications, honey bee-caused intoxications, wasp-caused intoxications, jelly fish-caused intoxications, scorpion-caused intoxications and spider-caused intoxications.

The term “plant-caused intoxication” as used herein refers to an intoxication, wherein the one or more external substance(s) exposed to the subject were produced a plant and transferred from the plant to the subject, e.g. by oral intake of the plant or a part of the plant.

Preferably, the, one or more or all external substance(s) is/are a protease, preferably selected from the group consisting of metalloproteases, cysteine proteases and serine proteases, preferably consisting of metalloproteases and serine proteases.

The tumor underlying the tumor disease may be any tumor. Preferably, the tumor is a tumor secreting one or more protease(s) selected from the group consisting of metalloproteases (preferably matrix metalloproteases), serine proteases, cysteine proteases, aspartate proteases or threonine proteases, preferably consisting of metalloproteases and serine proteases. Preferably, the tumor underlying the tumor disease is selected from the group consisting of breast cancer, colorectal cancer, pancreatic cancer, gastric cancer, prostate cancer, lung cancer, endometrial cancer, tongue carcinoma, laryngeal carcinoma, breast, lung, gastric, colon, pancreatic, head and neck carcinoma, Kaposi's sarcoma, atrophic gastritis with dysplasia, melanoma, glioma, adenocarcinoma and combinations thereof.

Measuring whether a tumor is secreting a protease as described above is performed by methods well known to a skilled person. For example, a tumor may be genotyped and the identified mutation may be associated with known pathways, at the end of which a metalloprotease (e.g. a matrix metalloprotease), a serine protease, a cysteine protease, an aspartate protease, a threonine protease or combinations thereof is produced and secreted. Moreover, for various cell types, it is known that metalloproteases (preferably matrix metalloproteases), serine proteases, cysteine proteases, aspartate proteases and/or threonine proteases are produced and secreted. Thus, in case such a cell is transformed into a tumor cell, the tumor also secretes a metalloprotease (preferably matrix metalloprotease), a serine protease, a cysteine protease, an aspartate protease, a threonine protease or combinations thereof. Additionally, it is known for distinct tumors that they secrete a metalloprotease (preferably matrix metalloprotease), a serine protease, a cysteine protease, an aspartate protease, a threonine protease or combinations thereof, as described above.

The tumor disease may be tumor metastasis. Thus, the term “prevention of tumor metastasis” is to be understood such that, preferably with regard to a particular tumor, no tumor metastasis has occurred yet in the subject and metastasis is prevented, e.g. by inhibiting or hampering the destruction of the basal membrane. Thus, it is preferably prevented that the tumor becomes a malign tumor. Further, the term “treatment of tumor metastasis” is to be understood such that, preferably with regard to a particular tumor, tumor metastasis has already occurred. However, the treatment of tumor metastasis prevents, e.g. inhibits or hampers, further tumor metastasis.

Preferably, the term “cysteine protease” includes cysteine proteases as well as cysteine-like proteases. Particularly preferably, the term refers to or includes a protease selected from the group consisting of cathepsins, preferably cathepsins B and L. Particularly preferably in the context of tumors as described herein, the term refers to or includes a protease selected from the group consisting of cathepsins, preferably cathepsins B and L.

Preferably, the term “serine protease” refers to or includes a protease selected from the group consisting of matriptase and trypsin. Particularly preferably in the context of tumors as described herein, the term refers to or includes a protease selected from the group consisting of thrombin, matriptase and trypsin.

Preferably, the term “aspartate protease” refers to or includes cathepsin D. Particularly preferably in the context of tumors as described herein, the term refers to or includes cathepsin D.

Preferably, the term “matrix metalloprotease” refers to or includes a protease selected from the group consisting of MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-21, MMP-23A, MMP-23B, MMP-24, MMP-25, MMP-26, MMP-27, MMP-28. Particularly preferably in the context of tumors as described herein, the term refers to or includes a protease selected from the group consisting of MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-21, MMP-23A, MMP-23B, MMP-24, MMP-25, MMP-26, MMP-27, MMP-28.

Preferably, the term “threonine protease” refers to or includes a protease selected from the group consisting of the 26S proteasome and TSP50. Particularly preferably in the context of tumors as described herein, the term refers to or includes a protease selected from the group consisting of the 26S proteasome and TSP50.

Additionally or alternatively to the proteases and preferred proteases described in the context of a tumor herein, it is preferred that the tumor secrets one or more protease(s) selected from the group consisting of ADAMs 8, 9, S1, and S13; cathepsins A, B, C, D, E, L, S, V, and X/Z/P; MMPs 1, 2, 3, 7, 8, 9, 10, 12, 13; kallikreins 5, 6, 7, 10, 11, 13; neprilysin/CD10, presenilin-1, PC-9, proteinase 3, and uPA.

With regard to the condensation product, component a1) may, for instance, be selected from the group consisting of phenol, ortho-cresol, meta-cresol, para-cresol, 2-ethylphenol, 3-ethylphenol, 4-ethylphenol, 2,3-5 dimethylphenol, 2,4-dimethylphenol, 2,5-dimethylphenol, 2,6-dimethylphenol, 3,4-dimethylphenol, 3,5-dimethylphenol, gallic acid, 9-hydroxyanthracene as a tautomer of anthrone, 9-hydroxyphenanthrene, diphenylmethane, phenyl-(2-methylphenyl)methane, phenylparatolylmethane, phenylmetatolylmethane.

Additionally or alternatively, component a2) may be selected from the group consisting of aldehydes and ketones, such as formaldehyde, acetaldehyde or propionaldehyde.

Additionally or alternatively, component a3) may be selected from the group consisting of sulphuric acid, chlorosulphonic acid, amidosulphonic acid, and oleum having an SO₃ content in the range of from 1 to 30% by weight.

Additionally or alternatively, component a4) may be selected from the group consisting of urea and derivatives thereof. Preferably, component a4) is selected from the group consisting of urea and a derivative thereof, wherein the derivative has a hydrogen atom on each nitrogen atom.

Advantageously, the condensation product to be used according to the present invention or a salt thereof is easy to produce, including its production in large amounts while its production costs are relatively low in comparison to other pharmaceutical agents. Thus, the condensation product to be used according to the invention can be widely and frequently be used for its applications. Furthermore, it might be comparatively easy to ensure its global distribution, especially its provision also for developing regions.

The term “Mw” as used herein refers to the weight-average molecular weight as determined by gel permeation chromatography (GPC) standard procedures, DIN standard 55672-1, using tetrahydrofuran (THF) as a solvent. Preferably, the weight-average molecular weight is determined by GPC with the following parameters:
Stationary phase: poly (2-hydroxymethacrylate) gel cross-linked with ethylene glycol dimethacrylate (commercially available as HEMA BIO from PSS, Mainz, Germany);
Eluent: Mixture of 30% by weight of tetrahydrofuran (THF), 10% by weight of acrylonitrile, 60% by weight of 1-molar NaNO3 solution, each related to the total weight of the mixture;
Internal standard: 0.001% by weight of benzophenone, based on the mobile phase;
Flow: 1.5 ml/min;
Concentration: 1% by weight, related to the eluent with the internal standard, in the eluent with the internal standard;
Detection: UV/Vis spectrometry at 254 nm;
Calibration with polystyrene calibration part of the company PSS.

Preferably, pharmaceutically acceptable carriers, as described herein, may be unpolar substances, preferably selected from the group consisting of polyacrylates, polymethacrylates, polyethers, polyesters, polyamides, polynitriles, etherificated celluloses, celluloseacetate, polynitrile, etherificated sugars and mixtures thereof.

Furthermore, the pharmaceutical composition may also comprise one or more additives, preferably selected from the group consisting of fillers, binders, lubricants, wetting agents, stabilizers, surfactants, preferably ionic surfactants and/or non-ionic surfactants, fatty alcohols and their esters, preferably alcoxylated fatty alcohols, and mixtures thereof, particularly preferably selected from the group consisting of unpolar substances, preferably selected from the group consisting of polyacrylates, polymethacrylates, polyethers, polyesters, polyamides, polynitriles, etherificated celluloses, celluloseacetate, polynitrile, etherificated sugars and mixtures thereof.

The term “phenolic compound” as used herein refers to a compound comprising one or more aromatic hydrocarbon group or groups bound directly to one or more hydroxyl groups (—OH).

The term “carbonyl compound” as used herein refers to a compound comprising one or more carbon atom or atoms, which is/are bound to an oxygen atom via a double bond (C═O).

The term “sulfonating agent” as used herein refers to a compound comprising one or more sulfonic acid functional group or groups and/or to one or more salt(s) thereof, such as sodium or potassium salts and mixtures thereof, which can thus serve as an educt in a sulfonation reaction.

The term “urea derivative” as used herein refers to any derivative of urea, preferably to a compound, which can be formed as a product of a chemical reaction involving urea as a precursor molecule.

Preferably, the condensation product or a salt thereof is produced by a method comprising the steps of
providing component a1)
providing component a2)
optionally: providing component a3) and/or component a4),.mixing components a1) and a2) by stirring, and applying a temperature of at most 105° C.,
stirring the obtained mixture at a temperature in the range of from 100 to 105° C. for a time in the range of from 0.5 to 5 hours
optionally: adding component a3) and/or component a4).

It is further preferred that component a1) is selected from the group consisting of phenol and dihydroxydiphenylsulfone.

Additionally or alternatively, component a2) is selected from the group consisting of formaldehyde, acetaldehyde and propionaldehyde.

Additionally or alternatively, component a3), if present, is sulfuric acid, preferably concentrated sulfuric acid.

Additionally or alternatively, component a4), if present, is selected from the group consisting of urea, melamine, the compound according to formula (I), the compound according to formula (II) and the compound according to formula (III),

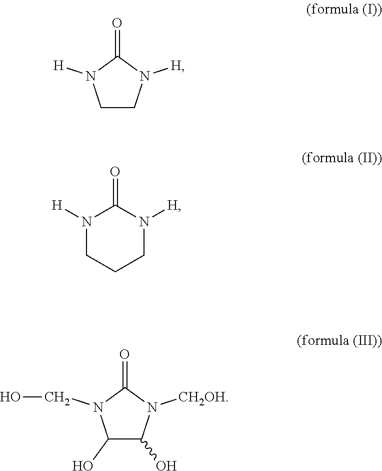

The term “concentrated sulphuric acid” as used herein refers to sulphuric acid in a liquid aggregate state and with a concentration of 90% or more, preferably 93% or more, more preferably 95% or more, even more preferably 97% or more, most preferably 99% or more.

Particularly, it is preferred that component a1) is phenol, component a2) is formaldehyde, component a3) is present and is sulfuric acid, preferably concentrated sulfuric acid, and component a4) is present and is urea.

Furthermore, it is preferred that the condensation product or salt thereof is or comprises a compound according to formula (IV), or is or comprises a salt of a compound according to formula (IV)

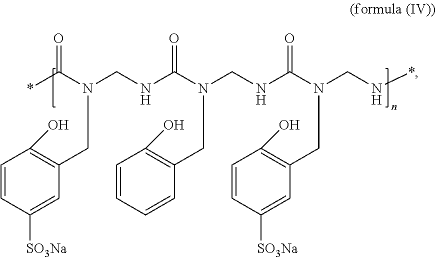

wherein n is an integer in a range of from 1 to 30, preferably in a range of from 2 to 25, particularly preferably in a range of from 3 to 20, further preferably in a range of from 4 to 15, more preferably in a range of from 5 to 12, especially preferably in a range of from 6 to 10, even further preferably in a range of from 7 to 9, or a derivative or pharmaceutically acceptable salt thereof.

Preferably, in the treatment or prevention of a condition associated with one or more protease(s) in a subject, the condensation product or the one or more salt(s) thereof or the condensation product and the one or more salt(s) thereof is/are administered at a daily dose in a range of from 0.005 to 10 mg/kg body weight of the subject, preferably in a range of from 0.0075 to 7.5 mg/kg body weight of the subject, preferably 0.01 to 5 mg/kg body weight of the subject, preferably in a range of from 0.025 to 2.5 mg/kg body weight of the subject, particularly preferably in a range of from 0.05 to 2 mg/kg body weight of the subject, especially preferably in a range of from 0.15 to 1 mg/kg body weight of the subject.

Preferably, the daily dose, as described herein, refers to the total dose of all condensation products according to the invention and all the salts thereof, as far as present.

Particularly preferably, the term “condensation product”, as used herein, describes the entirety of all present condensation products according to the invention, where applicable.

Thus, in case a parameter is to be determined for the condensation product, it is preferred that the entirety of all present condensation products are considered and included in the determination of said parameter. I.e. in case a daily dose of administration is to be determined, the dose is determined based on the entirety of all administered condensation products according to the invention.

Particularly preferably, in case a parameter is to be determined for the condensation product, it is preferred that the entirety of all present condensation products and the entirety of their present salts, as far as present, are considered and included in the determination of said parameter. I.e. in case a daily dose of administration is to be determined, the dose is determined based on the entirety of all administered condensation products according to the invention and all administered salts of condensation products according to the invention, as far as present.

Likewise, in case a condensation product is administered, the term “condensation product”, as used herein, preferably includes the entirety of all present condensation products according to the invention, particularly preferably the entirety of all present condensation products and the entirety of their present salts.

The term “daily dose” as used herein refers to an average daily amount per body weight of the condensation product as described herein or a salt thereof or the condensation product and a salt thereof, which is administered to a subject and does not necessarily require the administration to be performed every single day of a given time period. If the administration of the condensation product or a salt thereof or the condensation product and a salt thereof is performed once or more in a given number of days, the daily dose can be calculated by dividing the total administered amount of the condensation product or a salt thereof or the condensation product and a salt thereof per body weight of the subject by the respective given number of days.

Furthermore and preferably, the given number of days only describe a part of the treatments and/or preventions as described herein. Thus, in a first part of the treatments and/or preventions (i.e. a first given number of days), the daily dose may be different from a second, third, fourth, fifth or further part of the treatments and/or preventions (i.e. a second, third, fourth, fifth or further given number of days). Additionally or alternatively, the given number of days of the first, second, third, fourth, fifth and/or further part of the treatments and/or preventions may be different from the other part(s) of the treatments and/or preventions.

It is thus preferred, that the daily dose is higher in a first part of the treatment of a condition associated with one or more protease(s) in a subject, than in a second or further part.

It is thus preferred, that the daily dose is lower in a first part of treatment of a condition associated with one or more protease(s) in a subject, than in a second or further part.

It is thus preferred, that the daily dose is higher in a first part of the prevention of a condition associated with one or more protease(s) in a subject, than in a second or further part.

It is thus preferred, that the daily dose is lower in a first part of the prevention of a condition associated with one or more protease(s) in a subject, than in a second or further part.

Furthermore, it is preferred that the prevention and/or treatment, as described herein, further comprises the step of administering one or more further agent(s), selected from the group consisting of
antiviral agents, preferably wherein the, one or more or all of the antiviral agent(s) has/have an antiviral activity, preferably a virostatic or virucidal activity, against one, more or all of Cytomegalovirus (CMV), Human immunodeficiency virus (HIV), Dengue virus, Flavivirus, West-Nile virus (WNV), Chikungunya virus (CHIKV), Measles morbillivirus (MeV), Respiratory syncital virus (RSV) and Foot and Mouth disease virus;
antifungal agents, preferably wherein the, one or more or all of the antifungal agent(s) has/have an antifungal activity against one, more or all of pathogenic yeasts, dermatophytes and molds;
antibacterial agents, preferably wherein the, one or more or all of the antibacterial agent(s) has/have an antibacterial activity against one, more or all of Firmicutes and Propionibactericae;
anti-parasitic agents, preferably wherein the, one or more or all of the anti-parasitic agent(s) has/have an anti-parasitic activity against one, more or all of Shistosoma, Plasmodium, Trichinella, Toxoplasma, Trypanosoma, Leishmania, Trichomonas, Entamoeba and Giardia;
anti-allergic agents, preferably wherein the, one or more or all of the anti-allergic agent(s) is/are selected from the group consisting of anti-histamines and mast cell stabilizers;
antidotes, wherein the, one or more or all of the antidote(s) is/are selected from the group consisting of antidotes against one, more or all of snake venom, hymenoptera venom, jelly fish venom and arachnida venom;
anti-cancer agents, preferably wherein the, one or more or all of the anti-cancer agent(s) is/are selected from the group consisting of alkylating agents, anti-neoplastic antibiotics, antimetabolites, calcineurin-inhibitors, methotrexate, azathioprine, mTOR inhibitors, costimulator blockade and JAK inhibitors;
and combinations thereof.

The term “antiviral activity” as used herein refers to any activity, which reduces the entrance into a host cell, the replication, the spread, the infectivity, the expression of genes, the translation of mRNA, the packaging of proteins of a virus. Preferably, the, one or more or all antiviral agent(s) is/are selected from the group consisting of Abacavir, Aciclovir, Atazanavir, Camostat, Daclatasvir, Darunavir, Dasabuvir, Dolutegravir, Efavirenz, Emtricitabine, Entecavir, Famiciclovir, Foscarnet, Glecaprevir, Isoniazid, Lamivudine, Ledipasvir, Lopinavir, Nevirapine, Ombitasvir, Oseltamivir, Paritaprevir, Pegylated Interferon-Alpha-2a, Pegylated Interferon-Alpha-2b, Pibrentasvir, Pyridoxine, Raltegravir, Remdesivir, Ribavirin, Ritonavir, Sofosbuvir, Sulfamethoxazole, Tenofovir, Tenofovirdisoproxilfumarate, Trimethoprim, Valaciclovir, Valganciclovir, Velpatasvir and Zidovudine.

The term “antifungal activity” as used herein refers to any fungicidal and/or fungistatic activity, which reduces and/or inhibits fungal growth. Antifungal agents preferably reduce and/or inhibit the activity or expression of enzymes expressed by fungi, which leads to an inhibition of one or more biosynthesis pathways, such as for example biosynthesis of ergosterol and/or the biosynthesis of proteins and/or the biosynthesis of cell wall components. Additionally or alternatively, antifungal agents increase the general membrane permeability in fungi. Additionally or alternatively, antifungal agents reduce and/or inhibit nucleic acid synthesis, such as DNA synthesis in fungi. Additionally or alternatively, antifungal agents can facilitate formation of reactive oxygen species in fungi. Preferably, the, one or more or all antifungal agent(s) is/are selected from the group consisting of Amphotericin B, Clotrimazole, Fluconazole, Flucytosine, Griseofulvin, Itraconazole, Nystatin, Voriconazole and Potassiumiodide.

The term “antibacterial activity” as used herein refers to any bacteriostatic and/or bactericidal activity, which reduces and/or inhibits bacterial growth and/or kills bacteria. Antibacterial agents preferably reduce and/or inhibit the activity or expression of enzymes expressed by bacteria, resulting in a reduction or inhibition of the biosynthesis of cell wall components and/or in a reduction or inhibition of the biosynthesis of nucleic acids, such as for example DNA and/or RNA. Additionally or alternatively, antibacterial agents increase the general membrane permeability in bacteria. Additionally or alternatively, antibacterial agents can disrupt the cell wall of bacteria, preferably Gram-positive bacteria.

Preferably, the, one or more or all antibacterial agent(s) is/are selected from the group consisting of Amikacin, Amoxicillin, Amoxicillin, Ampicillin, Avibactam, Azithromycin, Bedaquiline, Benzathinebenzyl-Penicillin, Benzylpenicillin, Cefalexin, Cefazolin, Cefixime, Cefotaxime, Ceftazidime, Ceftriaxone, Cefuroxime, Chloramphenicol, Ciprofloxacin, Clarithromycin, Clavulanic Acid, Clindamycin, Clofazimine, Cloxacillin, Colistin, Cycloserine, Dapsone, Delamanid, Doxycycline, Ethambutol, Ethionamide, Fosfomycin, Gentamicin, Isoniazid, Levofloxacin, Linezolid, Meropenem, Metronidazole, Moxifloxacin, Nitrofurantoin, P-Aminosalicylicacid, Phenoxymethylpenicillin, Piperacillin, Plazomicin, Polymyxinb, Procainebenzylpenicillin, Pyrazinamide, Rifabutin, Rifampicin, Rifapentine, Spectinomycin, Streptomycin, Sulfamethoxazole, Tazobactam, Trimethoprim, Vaborbactam and Vancomycin.

The term “anti-parasitic activity” as used herein refers to any activity, which reduces and/or inhibits growth of parasitic organisms. Preferably, the, one or more or all anti-parasitic agent(s) is/are selected from the group consisting of Albendazole, Amodiaquine, Amphotericin B, Artemether, Artesunate, Benznidazole, Chloroquine, Diethylcarbamazine, Dihydroartemisinin, Diloxanide, Doxycycline, Eflornithine, Fexinidazole, Ivermectin, Levamisole, Lumefantrine, Mebendazole, Mefloquine, Meglumineantimoniate, Melarsoprol, Metronidazole, Miltefosine, Niclosamide, Nifurtimox, Oxamniquine, Paromomycin, Pentamidine, Piperaquinephosphate, Praziquantel, Primaquine, Proguanil, Pyrantel, Pyrimethamine, Pyronaridinetetraphosphate, Quinine, Sodiumstibogluconate, Sulfadiazine, Sulfadoxine, Sulfamethoxazole, Suraminsodium, Triclabendazole and Trimethoprim.

The term “antiallergic activity” as used herein refers to any activity, which reduces, inhibits, prevents or controls one or more symptoms of one/or more allergic disorder(s). As described above, the, one or more or all of the anti-allergenic agent(s) is/are preferably selected from the group consisting of anti-histamines and mast cell stabilizers. Preferably, the, one or more or all antiallergic agent(s) is/are selected from the group consisting of A-349, 821, ABT-239, Acrivastine, Alimemazine, Amitriptyline, Amoxapine, Aprotinin, Azelastine, Bilastine, Bromodiphenhydramine, Brompheniramine, Buclizine, Camostat, Carbinoxamine, Cetirizine, Chlorodiphenhydramine, Chloropyramine, Chlorpheniramine, Chlorpromazine, Chlorprothixene, Cimetidine, Cinnarizine, Ciproxifan, Clemastine, Clomipramine, Clozapine, Conessine, Cromoglicic acid, Cyclizine, Cyproheptadine, Desloratadine, Desloratidine, Dexamethasone, Dexbrompheniramine, Dexchlorpheniramine, Dimenhydrinate, Dimetindene, Diphenhydramine, Dosulepin, Doxepin, Doxylamine, E-64, Ebastine, Embramine, Epinephrine, Famotidine, Fexofenadine, Hydrocortisone, Hydroxyzine, Imipramine, JNJ 7777120, Ketofiten, Lafutidine, Levocabastine, Levocetirizine, Levomepromazine, Loratadine, Maprotiline, Meclizine, Mepolizumab, Methylxanthines, Mianserin, Mirtazapine, Nafamostat, Nedocromil, Nizatidine, Olanzapine, Olopatadine, Omalizumab, Orphenadrine, Palmitoylethanolamide, Pemirolast, Periciazine, Phenindamine, Pheniramine, Phenyltoloxamine, Prednisolone, Promethazine, Pyrilamine, Quercetin, Quetiapine, Ranitidine, Roxatidine, Rupatadine, Thioperamide, Tiotidine, Tranilast, Trazodone, Tripelennamine, Triprolidine, Vitamin D, VUF-6002 and β2-adrenergic agonists.

The term “antidote” as used herein refers to any substance, which can counteract an animal-caused intoxication or a plant-caused intoxication, as described above. Preferably the, one or more or all antidote(s) is/are antivenoms, preferably an antibodies. Particularly preferably, the antidote is selected from the group consisting of activated charcoal, Acetylcysteine, Atropine, Calcium gluconate, Methylthioninium chloride, Naloxone, Penicillamine, Prussian blue, Sodium nitrite, Sodium thiosulfate, Deferoxamine, Dimercaprol, Fomepizole, Sodium calcium edetate and Succimer.

The term “anti-cancer agent” as used herein refers to any molecule and/or compound and/or substance and/or mixture, which can have anti-carcinogenic activity. Preferably, the, one or more or all anti-cancer agent(s) is/are selected from the group consisting of alkylating agents, anti-neoplastic antibiotics, antimetabolites, calcineurin-inhibitors, methotrexate, azathioprine, mTOR inhibitors, costimulator blockade and JAK inhibitors.

Preferably, the, one or more or all anti-cancer agent(s) is/are selected from the group consisting of Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vemurafenib, Vinblastine, Vincristine and Vindesine.

Moreover, it is preferred that the pharmaceutical composition further comprises one or more further agent(s), selected from the group consisting of
antiviral agents, preferably wherein the, one or more or all of the antiviral agent(s) has/have an antiviral activity, preferably a virostatic or virucidal activity, against one, more or all of Cytomegalovirus (CMV), Human immunodeficiency virus (HIV), Dengue virus, Flavivirus, West-Nile virus (WNV), Chikungunya virus (CHIKV), Measles morbillivirus (MeV), Respiratory syncital virus (RSV) and Foot and Mouth disease virus;
antifungal agents, preferably wherein the, one or more or all of the antifungal agent(s) has/have an antifungal activity against one, more or all of pathogenic yeasts, dermatophytes and molds;
antibacterial agents, preferably wherein the, one or more or all of the antibacterial agent(s) has/have an antibacterial activity against one, more or all of Firmicutes and Propionibactericae;
anti-parasitic agents, preferably wherein the, one or more or all of the anti-parasitic agent(s) has/have an anti-parasitic activity against one, more or all of Shistosoma, Plasmodium, Trichinella, Toxoplasma, Trypanosoma, Leishmania, Trichomonas, Entamoeba and Giardia;
anti-allergic agents, preferably wherein the, one or more or all of the anti-allergic agent(s) is/are selected from the group consisting of anti-histamines and mast cell stabilizers;
antidotes, wherein the, one or more or all of the antidote(s) is/are selected from the group consisting of antidotes against one, more or all of snake venom, hymenoptera venom, jelly fish venom and arachnida venom;
anti-cancer agents, preferably wherein the, one or more or all of the anti-cancer agent(s) is/are selected from the group consisting of alkylating agents, anti-neoplastic antibiotics, antimetabolites, calcineurin-inhibitors, methotrexate, azathioprine, mTOR inhibitors, costimulator blockade and JAK inhibitors,
and combinations thereof.

What was said above with regard to the antiviral agents, antifungal agents, antibacterial agents, anti-parasitic agents, anti-allergic agents, antidotes, and anti-cancer agents applies accordingly, particularly with regard to preferred agents.

The present invention further relates to a pharmaceutical set comprising
(i) a pharmaceutical composition according to the invention and
(ii) one or more further agent(s), selected from the group consisting of
   (ii.a) antiviral agents, preferably wherein the, one or more or all of the antiviral agent(s) has/have an antiviral activity, preferably a virostatic or virucidal activity, against one, more or all of Cytomegalovirus (CMV), Human immunodeficiency virus (HIV), Dengue virus, Flavivirus, West-Nile virus (WNV), Chikungunya virus (CHIKV), Measles morbillivirus (MeV), Respiratory syncital virus (RSV) and Foot and Mouth disease virus;
   (ii.b) antifungal agent(s), preferably wherein the, one or more or all of the antifungal agent(s) has/have an antifungal activity against one, more or all of pathogenic yeasts, dermatophytes and molds;
   (ii.c) antibacterial agents, preferably wherein the, one or more or all of the antibacterial agent(s) has/have an antibacterial activity against one, more or all of Firmicutes and Propionibactericae;
   (ii.d) anti-parasitic agents, preferably wherein the, one or more or all of the anti-parasitic agent(s) has/have an anti-parasitic activity against one, more or all of Shistosoma, Plasmodium, Trichinella, Toxoplasma, Trypanosoma, Leishmania, Trichomonas, Entamoeba and Giardia;
   (ii.e) anti-allergic agent(s), preferably wherein the, one or more or all of the anti-allergic agent(s) is/are selected from the group consisting of anti-histamines and mast cell stabilizers;
   (ii.f) antidote(s), wherein the, one or more or all of the antidote(s) is/are selected from the group consisting of antidotes against one, more or all of snake venom, hymenoptera venom, jelly fish venom and arachnida venom;
   (ii.g) anti-cancer agent(s), wherein the, one or more or all of the anti-cancer agent(s) is/are selected from the group consisting of alkylating agents, anti-neoplastic antibiotics, antimetabolites, calcineurin-inhibitors, methotrexate, azathioprine, mTOR inhibitors, costimulator blockade and JAK inhibitors; and
   combinations thereof,
for use in a method for the treatment or prevention of a condition associated with one or more protease(s) in a subject,
wherein the condition is an infection with a pathogen expressing the one or more protease(s) or a reaction of the subject's organism to the one or more protease(s),
wherein the condition is selected from the group consisting of viral infections, fungal infections, bacterial infections, parasitoses, allergies, inflammatory conditions, intoxications and tumor diseases,
wherein the viral infections are selected from the group consisting of Cytomegalovirus (CMV) infections, Human immunodeficiency virus (HIV) infections, Dengue virus infections, Flavivirus infections, West-Nile virus (WNV) infections, Chikungunya virus (CHIKV) infections, Measles morbillivirus (MeV) infections, Respiratory syncital virus (RSV) infections and Foot and Mouth disease virus infections,
wherein the allergies are allergies against allergens selected from the group consisting of proteases,
   wherein the intoxications are animal-caused intoxications, preferably selected from snake-caused intoxications, hymenoptera-caused intoxications, jelly fish-caused intoxications and arachnida-caused intoxications.

Preferably, the tumor underlying the tumor disease is selected from tumors secreting one or more protease(s) selected from the group consisting of metalloproteases (preferably matrix metalloproteases), serine proteases, cysteine proteases, aspartate proteases or threonine proteases, preferably consisting of metalloproteases and serine proteases.

What was said above in the context of the condensation product or a salt thereof or, respectively, the pharmaceutical composition, applies accordingly for the pharmaceutical set, where applicable.

What was said above with regard to the proteases, antiviral agents, antifungal agents, antibacterial agents, anti-parasitic agents, anti-allergic agents, antidotes, and anti-cancer agents applies accordingly, particularly with regard to preferred agents.

What was said above with regard to the use of the condensation product to be used according to the invention, particularly with regard to preventions and treatments, infections, pathogens, reactions of the subject's organism described above, applies accordingly.

Preferably, components (i) and (ii) are present in a form separated from each other in the pharmaceutical set. Therefore, components (i) and (ii) may be present in separate containments, such as vials, tubes, bottles, cups, boxes or bags.

It is further preferred that the condensation product or salt thereof or pharmaceutical composition is present in a form selected from the group consisting of pills, tablets, lozenges, granules, capsules, preferably hard or soft gelatine capsules, aqueous solutions, alcoholic solutions, oily solutions, syrups, emulsions, suspensions, suppositories, pastilles, solutions for injection or infusion, ointments, tinctures, creams, lotions, powders, sprays, transdermal therapeutic systems, nasal sprays, aerosols, aerosol mixtures, microcapsules, implants, rods, patches and gels.

Particularly preferably, the condensation product or salt thereof or pharmaceutical composition is present in a form selected from the group consisting of aqueous solutions or sprays

With regard to a treatment and/or prevention as described herein, it is preferred that the administration is performed at an administration scheme of at least once in 14 day preferably at least once in 7 days, preferably at least once in 6 days, preferably at least once in 5 days, preferably at least once in 4 days, preferably at least once in 3 days, preferably at least once in 2 days, preferably at least once a day, preferably at least twice a day, preferably at least three times a day, preferably at least four times a day, preferably at least five times a day.

Preferably, the administration scheme is applied over a time period of at least 1 week, preferably at least 2 weeks, preferably at least 3 weeks, preferably at least 4 weeks, preferably at least 6 weeks, preferably at least 2 months, preferably at least 3 months, preferably at least 4 months, preferably at least 5 months, preferably at least 6 months, preferably at least 7 months, preferably at least 8 months, preferably at least 9 months, preferably at least 10 months, preferably at least 11 months, preferably at least 12 months, preferably at least 24 months.

Applying the administration scheme for a certain time period as described herein and within a certain regularity as described herein are not mutually exclusive. E.g., an administration of at least once in 7 days for at least 4 weeks describes the regularity of once in 7 days for a duration of at least 4 weeks or longer. The term “once in 7 days” as used herein is to be understood as one administration within a time frame of 7 days, wherein the administration may be at the beginning, the end or at any other time point within the 7 days. In case the administration is repeated, i.e. the time period of the administration scheme is longer than the administration interval, the time point of the respective administration (the beginning, the end or at any other time point within the interval) may vary between the respective administration intervals. E.g. in an administration of at least once in 7 days for at least 4 weeks, the administration in the first 7 days (first interval) may be performed at the beginning (day 1) of the interval and the administration in the second 7 days (second interval) may be performed on day 3 of the interval. The same applies accordingly to any other regularity and time period as described herein.

Preferably, the administration scheme in a first part of the treatments and/or preventions as described herein, the daily dose may be different from a second, third, fourth, fifth or further part of the treatments and/or preventions as described herein. E.g., the administration scheme in a first part of the treatments and/or preventions may be at least once a day, wherein in a second or further part of the treatments and/or preventions, the administration scheme may be once in two days.

It is thus preferred that the frequency of administration is higher in a first part of the treatment of a condition associated with one or more protease(s) as described herein, than in a second or further part.

It is thus preferred that the frequency of administration is lower in a first part of the treatment of a condition associated with one or more protease(s) as described herein, than in a second or further part.

It is thus preferred that the frequency of administration is higher in a first part of the prevention of a condition associated with one or more protease(s) as described herein, than in a second or further part.

It is thus preferred that the frequency of administration is lower in a first part of the prevention of a condition associated with one or more protease(s) as described herein, than in a second or further part.

Moreover, it is preferred that the step of administering is performed by or comprises an administration via an administration route selected from enteral or parenteral, preferably the step of administering is performed by or comprises an administration via an administration route selected from the group consisting of oral, nasal, intravenous, intra-arterial, inhalation, absorption over a mucous membrane and topical administration, preferably on the skin.

The present invention further relates to a pharmaceutical composition or pharmaceutical set comprising
(i) a condensation product to be used according to the invention or a salt thereof or the condensation product and a salt thereof and
(ii) one or more further agent(s), selected from the group consisting of
   (ii.a) antiviral agents, preferably wherein the, one or more or all of the antiviral agent(s) has/have an antiviral activity, preferably a virostatic or virucidal activity, against Cytomegalovirus (CMV), Human immunodeficiency virus (HIV), Dengue virus, Flavivirus, West-Nile virus (WNV), Chikungunya virus (CHIKV), Measles morbillivirus (MeV), Respiratory syncital virus (RSV) and Foot and Mouth disease virus;
   (ii.b) antifungal agent(s), preferably wherein the, one or more or all of the antifungal agent(s) has/have an antifungal activity against one, more or all of pathogenic yeasts, dermatophytes and molds;
   (ii.c) antibacterial agents, preferably wherein the, one or more or all of the antibacterial agent(s) has/have an antibacterial activity against one, more or all of Firmicutes and Propionibactericae;
   (ii.d) anti-parasitic agents, preferably wherein the, one or more or all of the anti-parasitic agent(s) has/have an anti-parasitic activity against one, more or all of Shistosoma, Plasmodium, Trichinella, Toxoplasma, Trypanosoma, Leishmania, Trichomonas, Entamoeba and Giardia;
   (ii.e) anti-allergic agent(s), preferably wherein the, one or more or all of the anti-allergic agent(s) is/are selected from the group consisting of anti-histamines and mast cell stabilizers;
   (ii.f) antidote(s), wherein the, one or more or all of the antidote(s) is/are selected from the group consisting of antidotes against one, more or all of snake venom, hymenoptera venom, jelly fish venom and arachnida venom;
   (ii.g) anti-cancer agent(s), preferably wherein the, one or more or all of the anti-cancer agent(s) is/are selected from the group consisting of alkylating agents, anti-neoplastic antibiotics, antimetabolites, calcineurin-inhibitors, methotrexate, azathioprine, mTOR inhibitors, costimulator blockade and JAK inhibitors; and
   combinations thereof.

What was said herein with regard to the condensation product or a salt thereof or, respectively, the pharmaceutical composition or the pharmaceutical set for use as described herein applies accordingly to the pharmaceutical composition or the pharmaceutical set independent of its use, where applicable.

What was said above with regard to the proteases, antiviral agents, antifungal agents, antibacterial agents, anti-parasitic agents, anti-allergic agents, antidotes, and anti-cancer agents applies accordingly, particularly with regard to preferred agents.

It is preferred that the pharmaceutical composition or one or more or all components of the pharmaceutical set is present in a form selected from the group consisting of pills, tablets, lozenges, granules, capsules, preferably hard or soft gelatine capsules, aqueous solutions, alcoholic solutions, oily solutions, syrups, emulsions, suspensions, suppositories, pastilles, solutions for injection or infusion, ointments, tinctures, creams, lotions, powders, sprays, transdermal therapeutic systems, nasal sprays, aerosols, aerosol mixtures, microcapsules, implants, rods, patches and gels,

Preferably, the pharmaceutical composition or one or more or all components of the pharmaceutical set is/are present in a form selected from the group consisting of aqueous solutions or sprays.

Preferably, the pharmaceutical composition or one or more or all components of the pharmaceutical set is/are present in a form selected from the group consisting of pills, tablets, aqueous solutions or sprays.

The present invention further relates to a non-therapeutic use of a condensation product or a salt thereof or a pharmaceutical composition or a pharmaceutical set,
as a disinfectant against pathogens selected from the group consisting of fungi, bacteria and parasites,
preferably wherein the fungi are selected from the group consisting of pathogenic yeasts, dermatophytes and molds,
preferably wherein the bacteria are selected from the group consisting of Firmicutes and Propionibactericae,
preferably wherein the parasites are selected from the group consisting of Shistosoma, Plasmodium, Trichinella, Toxoplasma, Trypanosoma, Leishmania, Trichomonas, Entamoeba and Giardia.

What was said above with regard to pathogens, particularly with regard to fungi, bacteria and parasites applies accordingly, especially with regard to preferred fungi, bacteria and parasites.

It was surprisingly found that the condensation product to be used according to the invention is capable of inhibiting the activity of proteases of pathogens, which are responsible or critically involved in the pathogen's ability to enter and/or reproduce in the subject's organism and/or which are critical in biological pathways essential for the pathogen.

Thus, applying the condensation product to be used according to the invention to pathogens, e.g. by spraying the condensation product to be used according to the invention or a composition comprising the condensation product to surfaces, which contain the pathogen(s), will inhibit the ability of a pathogen to enter and/or reproduce in the subject's organism or will inactivate or kill the pathogen already on the surface.

Thus, the condensation product to be used according to the invention can also be used (in a non-therapeutic way) as a disinfectant.

Further aspects and advantages of the invention result from the subsequent description of preferred examples.

### EXAMPLES

### Example 1: Producing a Condensation Product to be Used According to the Invention

Solutions are understood as meaning aqueous solutions if not expressly specified otherwise. ppm relates to parts by weight.

The molecular weight determinations are carried out using gel permeation chromatography (GPC):
Stationary phase: poly (2-hydroxymethacrylate) gel crosslinked with ethylene glycol dimethacrylate, obtainable commercially as HEMA BIO from PSS, Mainz, Germany.
Eluent: mixture of 30% by weight of tetrahydrofuran (THF), 10% by weight of acrylonitrile, 60% by weight of 1 molar NaNO3 solution
Internal standard: 0.001% by weight of benzophenone, based on eluent
Flow: 1.5 ml/min
Concentration: 1% by weight in the eluent containing internal standard
Detection: UV/Vis spectrometrically at 254 nm
Calibration using polystyrene calibration part from PSS.
Mn: number-average molecular weight in [g/mol]
Mw: weight-average molecular weight in [g/mol]

For the determination of free formaldehyde, a flow injection apparatus according to Huber is employed, see Fresenius Z. Anal. Chem. 1981, 309, 389. The column chosen is a thermostated reaction column 170×10 mm, filled with glass beads, which is operated at 75° C. The detector (continuous flow detector) is set at a wavelength of 412 nm. The procedure is as follows: For the preparation of a reagent solution, 62.5 g of ammonium acetate are dissolved in 500 ml of distilled water, 7.5 ml of concentrated acetic acid and 5.0 ml of acetylacetone are added and filled up to 1000 ml with distilled water. 0.1 g of the condensation product to be investigated is weighed into a 10 ml volumetric flask, filled up to 10 ml with distilled water and the respective sample solution is obtained. 100 μl of sample solution in each case are added, mixed with reagent solution and a mean residence time of 1.5 minutes is set, which corresponds to a flow of 35 ml/min.

For the determination of the absolute values, the flow injection apparatus is calibrated with formaldehyde solutions of known content.

### Example 1.1: Condensation Product A

Reactants were:
a1) phenol,
a2) concentrated sulfuric acid,
a3) formaldehyde,
a4) urea

2.04 kg of phenol are introduced into a stirring apparatus and treated with 2.48 kg of concentrated sulfuric acid (96% by weight) for 20 minutes. Care is to be taken here that the temperature does not exceed 105° C. Subsequently, the reaction mixture is stirred at 100 to 105° C. for 2 hours and then diluted with 0.34 kg of water of 20° C. and cooled to 70° C.

2.06 kg of aqueous urea solution (68% by weight) are metered in, the temperature rising to 95° C.; subsequently the mixture is cooled to 75° C.

4.10 kg of aqueous formaldehyde solution (30% by weight) are added over a period of 90 minutes, care being taken that the temperature does not rise above 75° C.

Subsequently, it is partially neutralized using 0.78 kg of aqueous sodium hydroxide solution (50% by weight), 0.30 kg of water are added, and the mixture is subsequently stirred for 30 minutes and cooled further.

1.36 kg of phenol are added at a temperature of 50° C. 1.14 kg of aqueous formaldehyde solution (30% by weight) are subsequently metered in at 50° C. over 20 minutes and the mixture is subsequently stirred for a further 30 minutes at 55° C.

The final adjustment of concentration and PH is carried out by addition of 1.40 kg of sodium hydroxide solution (50% by weight) and 2.5 kg of water. 18.5 kg of reaction solution 1.1 are obtained containing 43% by weight of non-volatile fractions.

The analysis of the reaction solution affords the following values:
sodium sulfate by IC (based on non-volatile fractions): 6.8% by weight;
phenol by HPLC (based on non-volatile fractions): 0.36% by weight;
4-phenolsulfonic acid by HPLC (based on non-volatile fractions): 2.89% by weight;
free formaldehyde: 75 ppm, based on non-volatile fractions;
Mn 890 g/mol, Mw 7820 g/mol, determined by GPC.

### Example 1.2: Condensation Product B

Reactants were
a) phenol,
b) concentrated sulfuric acid,
c) formaldehyde,
d) urea

2.04 kg of phenol are introduced into a stirring apparatus and treated with 2.48 kg of concentrated sulfuric acid (96% by weight) for 20 minutes. Care is to be taken here that the temperature does not exceed 105° C. Subsequently, the reaction mixture is stirred at 100 to 105° C. for 2 hours and then diluted with 340 g of water.

2.05 kg of urea solution (68% by weight) are metered in, care being taken that the temperature does not exceed 95° C.

3.60 kg of aqueous formaldehyde solution (30% by weight) are then added at 83 to 93° C. over a period of 1.5 hours.

After a stirring time of 15 minutes, 800 g of aqueous sodium hydroxide solution (50% by weight) are added, care being taken that the temperature does not exceed 85° C., so that the pH is subsequently between 7.3 and 7.5. 11.3 kg of reaction solution 1.2 containing 47% by weight of non-volatile fractions are obtained.

The analysis of reaction solution affords the following values:
sodium sulfate by IC (based on non-volatile fractions): 10.3% by weight;
phenol by HPLC (based on non-volatile fractions): 0.74% by weight;
4-phenolsulfonic acid by HPLC (based on non-volatile fractions): 1.36% by weight;
free formaldehyde: 99 ppm, based on non-volatile fractions;
Mn 1990 g/mol, Mw 17.020 g/mol, determined by GPC.

### Example 2: Protease Inhibition

### Example 2.1: Inhibition of Human Leukocyte Elastase (HLE) in Vitro

### Example 2.1.1

A condensation product was produced according to Example 1.2.

The condensation product, as well as two tea extracts were tested for their inhibitory properties against HLE in an in vitro enzyme activity test. The sample size was n=4.

The half maximal effective concentration (EC₅₀-value) was already achieved by low concentrations of the condensation product.

### Example 2.1.2

A condensation product was produced according to Example 1.1 (condensation product A). Furthermore, a condensation product was produced according to Example 1.2 (condensation product B).

The condensation products were tested for their inhibitory properties against HLE in an in vitro enzyme activity test. The sample size was n=4.

A half maximal effective concentration (EC₅₀-value) of 0.4 μg/ml was determined for condensation product A and a similar concentration was determined for condensation product B.

### Example 2.2: Inhibition of Proteinase 3 (PR3) in Vitro

### Example 2.2.1

A condensation product was produced according to Example 1.1 (condensation product A). Furthermore, a condensation product was produced according to Example 1.2 (condensation product B).

The condensation products as well as a glucose control were tested for their inhibitory properties against PR3 in an in vitro enzyme activity test. The sample size was n=4.

A half maximal effective concentration (EC₅₀-value)<1.0 g/ml was determined for condensation product A and a similar concentration was determined for condensation product B.

### Example 2.3: Inhibition of Murine Neutrophil Elastase (mNE) in Vitro

A condensation product was produced according to Example 1.2.

A solution containing mNE (10 μg/ml) and the condensation product (10 μg/ml) was incubated at 37° C. for 1 hour. Subsequently, in vitro enzyme activity assays were performed using (i) a FRET (Fluorescence Resonance Energy Transfer) active substrate with a recognition sequence of 8 amino acids and (ii) a thiobenzylic ester (sBzl) with a recognition sequence of 3 amino acids. Significant inhibiting effects of the condensation product against mNE could be determined for both of the tested substrates.

### Example 3: Antiviral Activity

### Example 3.1: Antiviral Activity Against Human Immunodeficiency Virus (HIV) in Vitro

### Example 3.1.1

A condensation product was produced according to Example 1.1 (condensation product A). Furthermore, a condensation product was produced according to Example 1.2 (condensation product B).

The condensation products were tested for their antiviral activity against HIV in vitro.

Cells were cultured and infected with HIV. Condensation product A or B was added and an inhibitory effect was measured and compared to a control sample, which was not treated with condensation product A or B.

The experiment was performed twice. In one approach, the respective condensation product was added when infecting the cells. In another approach, the cells were infected with HIV and the respective condensation product was added 24 h after infection. In both approaches, an inhibitory effect of the condensation products was observed.

A therapeutic index (TI) of approximately 300 was determined. The TI is defined as the median toxic concentration (TC₅₀) divided by the median inhibitory concentration (IC₅₀).

Both condensation products exceeded the required TI of 300. Condensation product A showed a TI of approximately 350, condensation product B showed an even higher TI.

### Example 3.1.2

A condensation product was produced according to Example 1.2.

A tea extract was provided.

Vero cells were infected with HIV type 1 (HIV-1) and treated with the condensation product or with the tea extract. The cytopathic effect (CPE) was determined 5 days post infection.

The condensation product showed an advantageous therapeutic effect on the infected Vero cells, with regard to CPE.

In contrast, a therapeutic index (TI) could not be detected for the tea extract.

### Example 3.2: Antiviral Activity Against Human Cytomegalovirus (HCMV) in Vitro

### Example 3.2.1

A condensation was produced according to Example 1.2.

The condensation product was tested for its antiviral activity against HCMV in vitro. A high antiviral activity of the condensation product against HCMV was observed.

### Example 3.2.2

A condensation product was produced according to Example 1.1 (condensation product A). Furthermore, a condensation product was produced according to Example 1.2 (condensation product B).

The condensation products were tested for their antiviral activity against HCMV in vitro. A therapeutic index (TI) of approximately 2000 was determined for both condensation products indicating a high antiviral activity of the condensation products against HCMV. The TI is defined as the median toxic concentration (TC₅₀) divided by the median inhibitory concentration (IC₅₀).

### Example 3.2.3

A condensation product was produced according to Example 1.2.

A tea extract was provided.

Human foreskin fibroblasts were infected with HCMV and treated with the condensation product or with the tea extract. The cytopathic effect (CPE) was determined 3 days post infection.

The condensation product showed an advantageous therapeutic effect on the infected fibroblasts, with regard to CPE.

In contrast, a therapeutic index (TI) could not be detected for the tea extract.

### Example 3.3: Antiviral Activity Against West-Nile Virus (WNV) in Vitro

A condensation product was produced according to Example 1.2.

A tea extract was provided.

Vero cells were infected with WNV and treated with the condensation product or the tea extract. The cytopathic effect (CPE) was determined 2 days post infection.

The condensation product showed an advantageous therapeutic effect on the infected Vero cells, with regard to CPE.

In contrast, a therapeutic index (TI) could not be detected for the tea extract.

### Example 3.4: Antiviral Activity Against Chikungunya Virus in Vitro

A condensation product was produced according to Example 1.2.

A tea extract was provided.

Vero cells were infected with Chikungunya virus and treated with the condensation product or the tea extract. The cytopathic effect (CPE) was determined 2 days post infection.

The condensation product showed an advantageous therapeutic effect on the infected Vero cells, with regard to CPE.

In contrast, a therapeutic index (TI) could not be detected for the tea extract.

### Example 3.5: Antiviral Activity Against Measles Morbillivirus in Vitro

A condensation product was produced according to Example 1.2.

A tea extract was provided.

Vero cells were infected with Measles morbillivirus and treated with the condensation product or the tea extract. The cytopathic effect (CPE) was determined 3 days post infection.

The condensation product showed an advantageous therapeutic effect on the infected Vero cells, with regard to CPE.

In contrast, a therapeutic index (TI) could not be detected for the tea extract.

### Example 3.6: Antiviral Activity Against Respiratory Syncital Virus in Vitro

A condensation product was produced according to Example 1.2.

A tea extract was provided.

Vero cells were infected with respiratory syncital virus and treated with the condensation product or the tea extract. The cytopathic effect (CPE) was determined 4 days post infection.

The condensation product showed an advantageous therapeutic effect on the infected Vero cells, with regard to CPE.

In contrast, a therapeutic index (TI) could not be detected for the tea extract.

Example 4: Antiallergic activity

| | Condensation product according |
|---|---|
| Allergen | to Example 1.2 (10 μg/ml) |
| Birch pollen | inhibition |
| Dermatophagoides pteronyssimus | inhibition |
| Dermatophagoides farinae | inhibition |

### Example 4.1: Antiallergic Activity Against Birch Pollen

A condensation product was produced according to Example 1.2.

A skin prick test was conducted using the condensation product (10 μg/ml) and testing its anti-allergic activity against birch pollen. A high rate of inhibition was determined in comparison to the untreated control.

### Example 4.2: Antiallergic Activity Against Dermatophagoides Pteronyssimus

A condensation product was produced according to Example 1.2.

A skin prick test was conducted using the condensation product (10 μg/ml) and testing its anti-allergic activity against Dermatophagoides pteronyssimus. A high rate of inhibition was determined in comparison to the untreated control.

### Example 4.3: Antiallergic Activity Against Dermatophagoides farinae

A condensation product was produced according to Example 1.2.

A skin prick test was conducted using the condensation product (10 μg/ml) and testing its anti-allergic activity against Dermatophagoides farinae. A high rate of inhibition was determined in comparison to the untreated control.

### Example 5: Anti-Tumor Activity

A condensation product was produced according to Example 1.2.

The ability of preventing angiogenesis was tested with the produced condensation product. Cells were treated with the condensation product at a concentration of 50 μl/ml. It was found that angiogenesis was reduced when compared to the untreated control.

1. A condensation product or salt thereof,
wherein the condensation product or salt thereof is obtained or obtainable by a reaction of
a1) phenol, and
a2) formaldehyde, and
a3) sulfuric acid, preferably concentrated sulfuric acid, and
a4) urea,
wherein the condensation product or salt thereof has a molecular weight M_{w} in the range of from 500 g/mol to 50,000 g/mol, and
optionally wherein the condensation product further comprises one or more pharmaceutically acceptable carrier.

2. The condensation product or salt thereof according to claim 1, wherein the condensation product or salt thereof comprises a compound according to formula (IV) 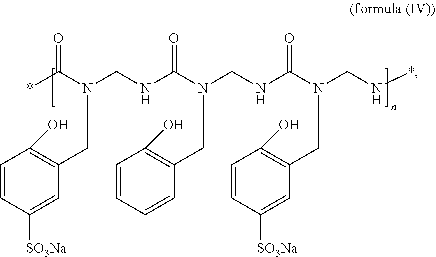
wherein n is an integer in a range of from 1 to 30,
or a derivative or pharmaceutically acceptable salt thereof.

3. The method according to claim 13, wherein the condensation product and/or the one or more salt thereof is administered at a daily dose in a range of from 0.005 to 10 mg/kg body weight of the subject.

4. The method according to claim 13, wherein the prevention and/or treatment further comprises administering one or more further agent, selected from the group consisting of
antiviral agents having a virostatic or virucidal activity against one or more of Cytomegalovirus (CMV), Human immunodeficiency virus (HIV), Dengue virus, Flavivirus, West-Nile virus (WNV), Chikungunya virus (CHIKV), Measles morbillivirus (MeV), Respiratory syncital virus (RSV), and Foot and Mouth disease virus;
antifungal agents having an antifungal activity against one or more of pathogenic yeasts, dermatophytes, and molds;
antibacterial agents having an antibacterial activity against one or more of Firmicutes and Propionibactericae;
anti-parasitic agents having an anti-parasitic activity against one or more of Shistosoma, Plasmodium, Trichinella, Toxoplasma, Trypanosoma, Leishmania, Trichomonas, Entamoeba, and Giardia;
anti-allergic agents, selected from the group consisting of anti-histamines and mast cell stabilizers;
antidotes selected from the group consisting of antidotes against one or more of snake venom, hymenoptera venom, jelly fish venom, and arachnida venom;
anti-cancer agents, selected from the group consisting of alkylating agents, anti-neoplastic antibiotics, antimetabolites, calcineurin-inhibitors, methotrexate, azathioprine, mTOR inhibitors, costimulator blockade, and JAK inhibitors; and
combinations thereof.

5. A pharmaceutical composition comprising the condensation product or salt thereof according to claim 1, wherein the pharmaceutical composition further comprises one or more further agent, selected from the group consisting of
antiviral agents having an antiviral activity against one or more of Cytomegalovirus (CMV), Human immunodeficiency virus (HIV), Dengue virus, Flavivirus, West-Nile virus (WNV), Chikungunya virus (CHIKV), Measles morbillivirus (MeV), Respiratory syncital virus (RSV), and Foot and Mouth disease virus;
antifungal agents having an antifungal activity against one or more of pathogenic yeasts, dermatophytes, and molds;
antibacterial agents having an antibacterial activity against one or more of Firmicutes and Propionibactericae;
anti-parasitic agents having an anti-parasitic activity against one or more of Shistosoma, Plasmodium, Trichinella, Toxoplasma, Trypanosoma, Leishmania, Trichomonas, Entamoeba, and Giardia;
anti-allergic agents selected from the group consisting of anti-histamines and mast cell stabilizers;
antidotes selected from the group consisting of antidotes against one or more of snake venom, hymenoptera venom, jelly fish venom, and arachnida venom;
anti-cancer agents selected from the group consisting of alkylating agents, anti-neoplastic antibiotics, antimetabolites, calcineurin-inhibitors, methotrexate, azathioprine, mTOR inhibitors, costimulator blockade, and JAK inhibitors; and
combinations thereof.

6. (canceled)

7. The pharmaceutical composition according to claim 5 in a form selected from the group consisting of pills, tablets, lozenges, granules, capsules, preferably hard or soft gelatine capsules, aqueous solutions, alcoholic solutions, oily solutions, syrups, emulsions, suspensions, suppositories, pastilles, solutions for injection or infusion, ointments, tinctures, creams, lotions, powders, sprays, transdermal therapeutic systems, nasal sprays, aerosols, aerosol mixtures, microcapsules, implants, rods, patches, and gels.

8. The method according to claim 13, wherein the administering is performed at an administration scheme of at least once in 14 days,
wherein the administration scheme is applied over a time period of at least 1 week.

9. The method according to claim 13, wherein the administering comprises an administration route selected from enteral or parenteral selected from the group consisting of oral, nasal, intravenous, intra-arterial, inhalation, absorption over a mucous membrane, and topical administration.

10. A pharmaceutical composition comprising
(i) a condensation product as defined in claim 1 and/or a salt thereof and
(ii) one or more further agent(s), selected from the group consisting of
(ii.a) antiviral agents having an antiviral activity against Cytomegalovirus (CMV), Human immunodeficiency virus (HIV), Dengue virus, Flavivirus, West-Nile virus (WNV), Chikungunya virus (CHIKV), Measles morbillivirus (MeV), Respiratory syncital virus (RSV) and Foot, and Mouth disease virus;
(ii.b) antifungal agent(s) having an antifungal activity against one or more of pathogenic yeasts, dermatophytes, and molds;
(ii.c) antibacterial agents having an antibacterial activity against one or more of Firmicutes and Propionibactericae;
(ii.d) anti-parasitic agents having an anti-parasitic activity against one or more of Shistosoma, Plasmodium, Trichinella, Toxoplasma, Trypanosoma, Leishmania, Trichomonas, Entamoeba, and Giardia;
(ii.e) anti-allergic agent(s) selected from the group consisting of anti-histamines and mast cell stabilizers;
(ii.f) antidote(s) selected from the group consisting of antidotes against one or more of snake venom, hymenoptera venom, jelly fish venom, and arachnida venom;
(ii.g) anti-cancer agent(s) selected from the group consisting of alkylating agents, anti-neoplastic antibiotics, antimetabolites, calcineurin-inhibitors, methotrexate, azathioprine, mTOR inhibitors, costimulator blockade, and JAK inhibitors; and
combinations thereof.

11. The pharmaceutical composition according to claim 10 in a form selected from the group consisting of pills, tablets, lozenges, granules, capsules, preferably hard or soft gelatine capsules, aqueous solutions, alcoholic solutions, oily solutions, syrups, emulsions, suspensions, suppositories, pastilles, solutions for injection or infusion, ointments, tinctures, creams, lotions, powders, sprays, transdermal therapeutic systems, nasal sprays, aerosols, aerosol mixtures, microcapsules, implants, rods, patches, and gels.

12. A disinfectant comprising a condensation product or salt thereof as defined in claim 1,
wherein the disinfectant is effective against pathogens selected from the group consisting of
fungi selected from the group consisting of pathogenic yeasts, dermatophytes, and molds,
bacteria selected from the group consisting of Firmicutes and Propionibactericae, and
parasites selected from the group consisting of Shistosoma, Plasmodium, Trichinella, Toxoplasma, Trypanosoma, Leishmania, Trichomonas, Entamoeba, and Giardia

13. A method for treating or preventing a condition associated with one or more protease in a subject comprising administering a condensation product according to claim 1 to the subject,
wherein the condition is an infection with a pathogen expressing the one or more protease or a reaction of the subject to the one or more protease,
wherein the condition is selected from the group consisting of viral infections, fungal infections, bacterial infections, parasitoses, allergies, inflammatory conditions, intoxications, and tumor diseases,
wherein the viral infections are selected from the group consisting of Cytomegalovirus (CMV) infections, Human immunodeficiency virus (HIV) infections, Dengue virus infections, Flavivirus infections, West-Nile virus (WNV) infections, Chikungunya virus (CHIKV) infections, Measles morbillivirus (MeV) infections, Respiratory syncital virus (RSV) infections and Foot, and Mouth disease virus infections,
wherein the allergies are directed against allergens selected from the group consisting of proteases,
wherein the intoxications are selected from the group consisting of (a) animal-caused intoxications selected from the group consisting of snake-caused intoxications, hymenoptera-caused intoxications, jelly fish-caused intoxications, arachnida-caused intoxications, and (b) plant-caused intoxications.
